(19) United States
(12) Patent Application Publication
(10) Pub. No.: US 2016/0051200 A1
(43) Pub. Date: Feb. 25, 2016

(54) MEDICAL IMAGING SYSTEM, MEDICAL IMAGING METHOD, AND NON-TRANSITORY COMPUTER-READABLE STORAGE MEDIUM WHICH STORES A MEDICAL IMAGING PROGRAM

(71) Applicant: ZIOSOFT, INC., Tokyo (JP)

(72) Inventor: Shusuke CHINO, Tokyo (JP)

(73) Assignee: ZIOSOFT, INC., Tokyo (JP)

(21) Appl. No.: 14/783,718

(22) PCT Filed: Apr. 10, 2014
(86) PCT No.: PCT/JP2014/060362

§ 371 Date: Oct. 9, 2015

(30) Foreign Application Priority Data
Apr. 11, 2013 (JP) 2013-083420

(51) Int. Cl. A61B 5/00; A61B 6/03; G06T 15/00; A61M 5/00; G06T 11/20; A61B 5/055; A61B 6/00

(52) U.S. Cl. CPC A61B 5/72; A61B 5/055; A61B 6/032; A61B 6/481; A61B 5/0033; A61B 5/743; A61M 5/007; G06T 11/206; G06T 15/005; G06T 2200/04

(57) ABSTRACT

There is provided a medical imaging system, a medical imaging method, and a medical imaging program capable of easily checking the imaging timing of an image and the injection timing of a contrast agent. The medical imaging system includes: a slice image acquisition unit that acquires a set of slice images of a subject with an image management information for managing the slice images; an injection information acquisition unit that acquires contrast agent injection information that is information regarding a contrast agent; an injection progress imaging unit that generates an injection progress image showing an injection progress of the contrast agent based on the contrast agent injection information; an association constructing unit that associates the contrast agent injection information with the slice image based on the contrast agent injection information and the image management information; and an output unit that outputs an slice image from the set of slice images and that outputs the contrast agent injection information associated with the slice image, the injection progress image generated based on the contrast agent injection information, and the image management information corresponding to the slice image, so as to be superimposed on each other.

### TECHNICAL FIELD

The present invention relates to a medical imaging system, a medical imaging method, and a medical imaging program. In particular, the present invention relates to a medical imaging system, a medical imaging method, and a medical imaging program capable of associating the imaging timing of a slice image of a subject with information regarding the injection timing of a contrast agent.

### BACKGROUND ART

Conventionally, an image processing apparatus has been known that acquires three-dimensional volume data from an imaging apparatus, designates blood vessel sectional regions sequentially from the starting point of the blood vessel to be extracted, makes a blood vessel section in each sectional region visible on an image based on the change rate of the area of an image that appears according to a change in the threshold value in each of the designated blood vessel sectional regions, calculates a center position of the visualized blood vessel section, and lists a plurality of kinds of images showing the target blood vessel, which is generated based on a three-dimensional path indicating a longitudinal direction center line of the target blood vessel, on a display device (for example, refer to Patent Document 1). According to the image processing apparatus disclosed in Patent Document 1, it is possible to accurately extract the tubular tissue, such as a blood vessel, in a medical image.

### CITATION LIST

### Patent Documents

Patent Document 1: Japanese Patent Application Laid-Open No. 2004-313736

### SUMMARY OF THE INVENTION

### Problems to be Solved by the Invention

In various medical image processing apparatuses including the image processing apparatus disclosed in Patent Document 1, it is thought that being able to easily check the imaging timing of an image and the timing of injection of a contrast agent, which is used when capturing the image, into a subject is helpful for more accurate diagnosis of the subject by a doctor or the like.

However, the imaging apparatus and the injector for injecting the contrast agent are different apparatuses. In normal cases, therefore, a radiologist makes a note of the contrast agent injection time in the injector, and the note is checked when observing the volume data acquired from the imaging apparatus later. In this method, however, it is difficult to determine how much the contrast affects the volume data.

Therefore, it is an object of the present invention to provide a medical imaging system, a medical imaging method, and a medical imaging program capable of easily checking the imaging timing of an image and the injection timing of a contrast agent.

### Means for Solving the Problems

In order to achieve the object described above, according to an aspect of the present invention, a medical imaging system includes: a slice image acquisition unit that acquires a set of slice images of a subject with an image management information for managing the slice image; an injection information acquisition unit that acquires contrast agent injection information that injected into the subject; an injection progress imaging unit that generates an injection progress image showing an injection progress of the contrast agent based on the contrast agent injection information; an association constructing unit that associates the contrast agent injection information with the slice image based on the contrast agent injection information and the image management information; and an output unit that outputs an slice image from the set of slice images and that outputs the contrast agent injection information associated with the slice image, the injection progress image generated based on the contrast agent injection information, and the image management information corresponding to the slice image, so as to be superimposed on each other.

In the medical imaging system described above, the injection progress imaging unit may generate the injection progress image using injection time data of the contrast agent and injection pressure data of the contrast agent. When a designation of a predetermined time is received from outside, the output unit may output a slice image captured at the time. When a designation of a predetermined slice image is received from outside, the output unit may output an imaging time of the slice image.

The medical imaging system described above may further include an information addition unit that adds the image management information, which corresponds to the slice image associated with the contrast agent injection information, to the contrast agent injection information.

In the medical imaging system described above, the output unit may output time series information relevant to capturing of the slice image based on the contrast agent injection information, the slice image associated with the contrast agent injection information, and the image management information corresponding to the slice image.

The medical imaging system described above may further include a storage unit that stores the slice image corresponding to the image management information, the contrast agent injection information, and the injection progress image generated based on the contrast agent injection information.

The medical imaging system described above may further include a three-dimensional imaging unit that generates a three-dimensional image from the slice image, and the output unit may output the three-dimensional image.

In order to achieve the object described above, according to another aspect of the present invention, a medical imaging method in an image processing apparatus, the method includes: a slice image acquisition step of acquiring a set of slice images of a subject with an image management information for managing the slice images; an injection information acquisition step of acquiring contrast agent injection information that injected into the subject; an injection progress imaging step of generating an injection progress image showing an injection progress of the contrast agent based on the contrast agent injection information; an association constructing step of associating the contrast agent injection information with the slice image based on the contrast agent injection information and the image management information; and an output step of outputting an slice image from the set of slice images and outputting the contrast agent injection information associated with the slice image, the injection progress image generated based on the contrast agent injection information, and the image management information corresponding to the slice image, so as to be superimposed on each other.

In order to achieve the object described above, according to still another aspect of the present invention, there is provided a medical imaging program for generating a medical image. The medical imaging program causes a computer to realize: a slice image acquisition function of acquiring a set of slice images of a subject with an image management information for managing the slice images; an injection information acquisition function of acquiring contrast agent injection information that injected into the subject; an injection progress imaging function of generating an injection progress image showing an injection progress of the contrast agent based on the contrast agent injection information; an association constructing function of associating the contrast agent injection information with the slice image based on the contrast agent injection information and the image management information; and an output function of outputting an slice image from the set of slice images and outputting the contrast agent injection information associated with the slice image, the injection progress image generated based on the contrast agent injection information, and the image management information corresponding to the slice image, so as to be superimposed on each other.

### Advantageous Effect of the Invention

According to the medical imaging system, the medical imaging method, and the medical imaging program according to the present invention, it is possible to provide a medical imaging system, a medical imaging method, and a medical imaging program capable of easily checking the imaging timing of an image and the injection timing of the contrast agent.

### BRIEF DESCRIPTION OF THE DRAWINGS

FIG. 1 is a schematic diagram of a medical imaging system according to the present embodiment.

FIG. 2 is a functional block diagram of the medical imaging system according to the present embodiment.

FIG. 3 is a diagram illustrating an output result in an output unit of the medical imaging system according to the present embodiment.

FIG. 4 is an enlarged view of an injection progress image illustrated in FIG. 3.

FIG. 5 is an enlarged view of the injection progress image illustrated in FIG. 3.

FIG. 6 is a flow diagram of the medical imaging system according to the embodiment of the present invention.

FIG. 7 is a hardware configuration diagram of the medical imaging system according to the present embodiment.

### MODE FOR CARRYING OUT THE INVENTION

### Embodiments

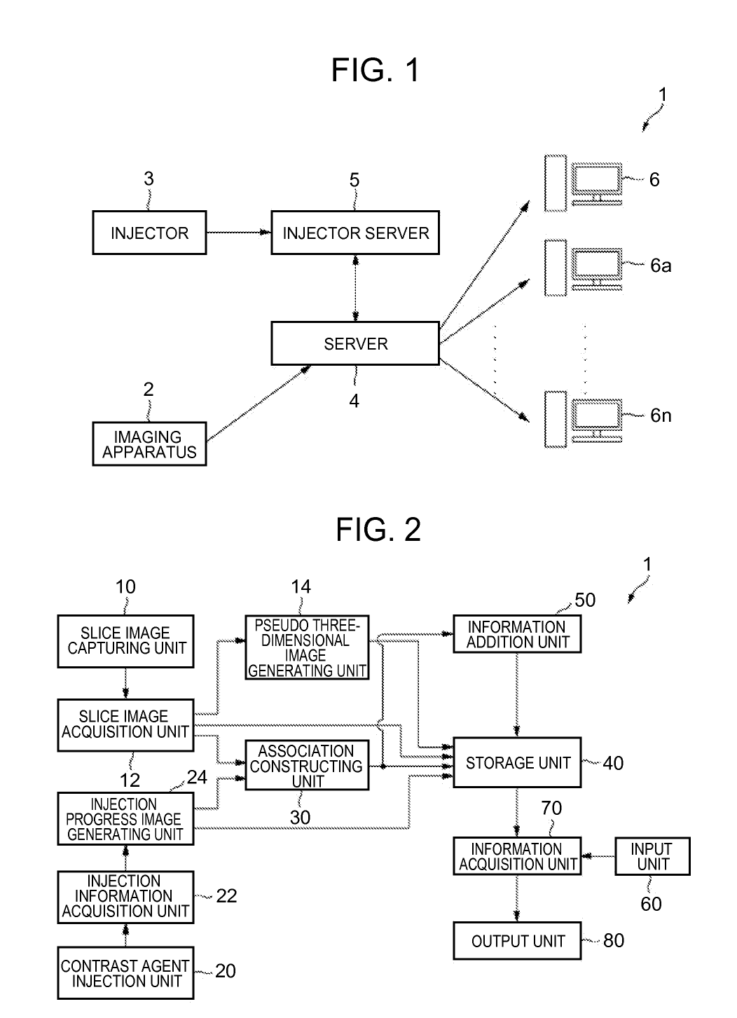

FIG. 1 illustrates an example of the outline of a medical imaging system according to the present embodiment.

(Outline of a Medical Imaging System 1)

The medical imaging system 1 according to the present embodiment acquires a set of slice images of a patient as a subject into which a contrast agent has been injected, as image data, together with image management information corresponding to the image data, and automatically associates contrast agent injection information, which is information regarding the injection of a contrast agent into the patient, with the image data based on the image management information. Then, when outputting the slice image to a monitor of an terminal 6 or the like, the medical imaging system 1 outputs both the contrast agent injection information, which is associated with the image data of the slice image, and the image management information (for example, information regarding the time when the slice image has been captured).

Specifically, as illustrated in FIG. 1, the medical imaging system 1 includes an imaging apparatus 2 that captures images of a patient as a subject, an injector 3 for injecting a contrast agent into the patient, a server 4 for storing image data captured by the imaging apparatus 2 and information supplied from an injector server 5, and the injector server 5 that acquires contrast agent injection information when the contrast agent is injected into the patient by the injector 3. In addition, the medical imaging system 1 includes terminals 6, 6a, . . . , and 6n. The system can acquire the image data, the image management information, and the contrast agent injection information from the server 4 and can output the contrast agent injection information and the image management information so as to be superimposed on each other. Here, the server 4 or the injector server 5 automatically associates the contrast agent injection information with the image data. Although a plurality of terminals is illustrated in FIG. 1, the medical imaging system 1 can also be configured to include only one terminal 6.

As an example, in the medical imaging system 1, the imaging apparatus 2 captures a set of two-dimensional slice image of the patient, and the injector 3 injects a contrast agent into the patient. Then, the set of slice images is supplied to the server 4 together with the image management information corresponding to the image data. On the other hand, the contrast agent injection information including information regarding the time when the contrast agent has been injected into the patient by the injector 3 and the like is supplied to the injector server 5. Then, the server 4 or the injector server 5 compares the imaging time when the slice image has been captured with the start time of the injection of contrast agent into the patient. Then, the server 4 or the injector server 5 associates the image data and the contrast agent injection information in which the imaging time and the injection start time substantially match each other.

Then, the medical imaging system 1 outputs two-dimensional slice images of the patient and a three-dimensional image, which is generated using a plurality of two-dimensional slice images (as a volume data), on the monitor of the terminal 6 or the like. Here, the medical imaging system 1 receives an input from the outside, and outputs collectively contrast agent injection information and image management information (for example, information indicating the imaging start time, imaging continuation time, and the like) at the time when a two-dimensional slice image or a three-dimensional image of an arbitrary affected part has been captured. Therefore, since the medical imaging system 1 can visually inform a user using the medical imaging system 1 of, for example, a correlation between the injection timing of the contrast agent and the duration of injection and the imaging timing of an image, it is possible to provide the user with useful information for the diagnosis of a patient.

The imaging apparatus 2 is a computed tomography (CT) apparatus, a magnetic resonance imaging system (MRI apparatus), or the like. In the present embodiment, the image management information is information based on the standards defining the data format and a communication method of information regarding a captured image that is captured by the CT, MRI, or the like. For example, the image management information is DICOM tag information based on the digital imaging and communications in medicine (DICOM) standards. The server 4 is a server that manages the image management information. For example, the server 4 is a DICOM server.

(Configuration of the Medical Imaging System 1)

FIG. 2 illustrates an example of the functional configuration of a medical imaging system according to the present embodiment.

(Outline of the Configuration of the Medical Imaging System 1)

The medical imaging system 1 includes: a slice image capturing unit 10 that captures a slice image of a subject; a slice image acquisition unit 12 that acquires the slice image so as to correspond to image management information; a three-dimensional imaging unit 14 that generates a three-dimensional image from the slice image; a contrast agent injection unit 20 that injects a contrast agent into the subject; an injection information acquisition unit 22 that acquires contrast agent injection information, which is information regarding the injection of a contrast agent, from the contrast agent injection unit 20; an injection progress imaging unit 24 that generates an injection progress image showing the injection status of the contrast agent based on the contrast agent injection information; an association constructing unit 30 that associates the contrast agent injection information with the slice image based on the contrast agent injection information and the image management information; and a storage unit 40 that stores the slice image corresponding to the image management information, the contrast agent injection information, and the injection progress image generated based on the contrast agent injection information.

In addition, the medical imaging system 1 includes: an information addition unit 50 that adds the image management information, which corresponds to the slice image associated with the contrast agent injection information, to the contrast agent injection information; an input unit 60 that receives a slice image output instruction from the outside; an information acquisition unit 70 that acquires the slice image, the contrast agent injection information associated with the slice image, and the injection progress image generated from the contrast agent injection information, from the storage unit 40 when the input unit 60 receives an output instruction; and an output unit 80 that outputs the slice image acquired by the information acquisition unit 70 and that outputs the contrast agent injection information and the injection progress image acquired by the information acquisition unit 70 and the image management information corresponding to the slice image so that the contrast agent injection information and the injection progress image and the image management information are superimposed on each other.

As an example, the imaging apparatus 2 illustrated in FIG. 1 includes at least the slice image capturing unit 10. The injector 3 includes at least the contrast agent injection unit 20. The server 4 includes the slice image acquisition unit 12, the three-dimensional imaging unit 14, the storage unit 40, and the information addition unit 50. The injector server 5 includes the injection information acquisition unit 22. The server 4 or the injector server 5 can include the injection progress imaging unit 24 and the association constructing unit 30. The terminal 6 includes the input unit 60, the information acquisition unit 70, and the output unit 80.

(Slice Image Capturing Unit 10)

The slice image capturing unit 10 captures a slice image of a patient who is a subject. For example, the slice image capturing unit 10 includes a radiation source for emitting radiation to the subject, a radiation detector that detects radiation having passed through the subject after a part of the radiation emitted to the subject from the radiation source is absorbed by the subject and that is disposed at a symmetrical position of the radiation source with respect to the subject, and an information processing device that forms image data of the slice image from the detection result of the radiation detector. Then, the slice image capturing unit 10 can capture a set of slice images in a single scan operation. The slice image capturing unit 10 supplies the image data of the captured slice image to the slice image acquisition unit 12.

(Slice Image Acquisition Unit 12)

The slice image acquisition unit 12 acquires a slice image from the slice image capturing unit 10 so as to correspond to the image management information for managing the slice images of the subject. The slice image acquisition unit 12 acquires a set of slice images, which are captured in a single scan operation (or one examination) by the slice image capturing unit 10, as volume data. The slice image acquisition unit 12 can also associate the volume data with a series number. The slice image acquisition unit 12 supplies image data of the slice image, which corresponds to the image management information, to the three-dimensional imaging unit 14, the association constructing unit 30, and/or the storage unit 40. Not only does the slice image form volume data, but also the slice image may form time-series slice image data that is captured in time series. In addition, volume data may be obtained by performing imaging multiple times in time series. For example, volume data including a plurality of phases may be obtained by imaging the process of the beating of the heart that is ECG-synchronized.

The image management information according to the present embodiment includes information indicating an examination identifier for uniquely identifying the examination of the subject by image capturing, an imaging date when a slice image has been captured, an imaging start time that is a time when the imaging of the slice image has been started, a name of the subject (for example, when the subject is a patient, a patient name), a subject identifier for uniquely identifying the subject, date of birth of the subject, age of the subject, sex of the subject, an examination part of the subject, and the like. For example, the image management information is a DICOM tag.

(Three-Dimensional Imaging Unit 14)

The three-dimensional imaging unit 14 generates a three-dimensional image from the set of slice images acquired by the slice image acquisition unit 12. Here, the three-dimensional image is a stereoscopic image generated using a set of slice images. The three-dimensional imaging unit 14 associates the generated three-dimensional image with the image management information (for example, a subject identifier). Then, the three-dimensional imaging unit 14 supplies the three-dimensional image to the storage unit 40.

(Contrast Agent Injection Unit 20)

The contrast agent injection unit 20 injects a contrast agent into the subject. When injecting a contrast agent into the subject, the contrast agent injection unit 20 acquires contrast agent injection information. The contrast agent injection information is information indicating the injection time of the contrast agent, the injection duration of the contrast agent, the injection pressure of the contrast agent, the injection rate of the contrast agent, the injection amount of the contrast agent, the amount of iodine, and/or the type of the contrast agent. The contrast agent injection unit 20 supplies the contrast agent injection information to the injection information acquisition unit 22. In the present embodiment, the contrast agent injection unit 20 does not need to associate the identifier for identifying the subject with the contrast agent injection information during the injection of the contrast agent. Then, the contrast agent injection unit 20 supplies the contrast agent injection information to the injection information acquisition unit 22. Therefore, in the present embodiment, it is not necessary to input the information of the name of a patient as a subject to the injector 3.

(Injection Information Acquisition Unit 22)

The injection information acquisition unit 22 acquires contrast agent injection information from the contrast agent injection unit 20. The injection information acquisition unit 22 supplies the acquired contrast agent injection information to the injection progress imaging unit 24. The injection information acquisition unit 22 can also directly supply the acquired contrast agent injection information to the association constructing unit 30 and the storage unit 40.

(Injection Progress Imaging Unit 24)

The injection progress imaging unit 24 generates an injection progress image showing the injection progress of the contrast agent based on the contrast agent injection information. Specifically, the injection progress imaging unit 24 generates an injection progress image using the contrast agent injection time data and the contrast agent injection pressure data. For example, the injection progress image is a graph showing a temporal change in the injection pressure or the injection amount of the contrast agent that is obtained by setting the time on the horizontal axis and the injection pressure or the injection amount of the contrast agent on the vertical axis. The injection progress imaging unit 24 supplies the generated injection progress image to the association constructing unit 30 and the storage unit 40.

(Association Constructing Unit 30)

The association constructing unit 30 associates the contrast agent injection information with the image data of the slice image based on the contrast agent injection information and the image management information. Specifically, the association constructing unit 30 compares data indicating the injection time of the contrast agent, which is included in the contrast agent injection information, and data indicating the imaging time at which the slice image has been captured, which is included in the image management information. When the injection time and the imaging time are substantially the same or when the imaging time is set as a reference time and the injection time is present within a predetermined time from the set reference time, the association constructing unit 30 associates the contrast agent injection information at the injection time with the slice image captured at the imaging time. In addition, when the injection time is set as a reference time and the imaging time is present within a predetermined time from the set reference time, the association constructing unit 30 may associate the contrast agent injection information at the injection time with the slice image captured at the imaging time. The association constructing unit 30 supplies the slice image associated with the contrast agent injection information to the storage unit 40. In addition, the association constructing unit 30 supplies the slice image to the storage unit 40 together with some of the image management information associated with the injection progress image. For example, it is possible to acquire a DICOM tag, which enables the identification of imaging, from the DICOM tag of the image management information and attach the DICOM tag to the injection progress image.

(Storage Unit 40)

The storage unit 40 stores the slice image corresponding to the image management information, the contrast agent injection information, and the injection progress image generated based on the contrast agent injection information. The storage unit 40 can also store the three-dimensional image generated by the three-dimensional imaging unit 14. The storage unit 40 supplies the slice image, the contrast agent injection information, the injection progress image, and the three-dimensional image, which are stored, to the information acquisition unit 70 in response to the request of the information acquisition unit 70.

(Information Addition Unit 50)

The information addition unit 50 adds image management information, which corresponds to the slice image associated with the contrast agent injection information, to the contrast agent injection information. For example, the contrast agent injection information in the present embodiment can be made as information in a form that conforms to the DICOM. Then, the information addition unit 50 inserts the image management information, such as an examination identifier, into the DICOM tag of the contrast agent injection information. As an example, the information addition unit 50 adds the image management information to the private tag of the DICOM.

In addition, the information addition unit 50 can also insert a series number into the contrast agent injection information, for example. By adding the image management information to the contrast agent injection information, the medical imaging system 1 can use a piece of contrast agent injection information and image management information and a slice image, which are associated with a piece of contrast agent injection information, as information acquired in the same examination. The information addition unit 50 stores the contrast agent injection information, which includes the image management information that has been added, in the storage unit 40.

(Input Unit 60)

The input unit 60 receives an instruction of the user from the outside. For example, the input unit 60 receives a slice image output instruction, designation of the imaging time of a slice image, and the like from the outside. The input unit 60 supplies the received instruction to the information acquisition unit 70.

(Information Acquisition Unit 70)

The information acquisition unit 70 acquires predetermined information from the storage unit 40 when the input unit 60 receives a predetermined instruction. For example, when the input unit 60 receives a slice image output instruction, the information acquisition unit 70 acquires a designated slice image, contrast agent injection information associated with the slice image, and an injection progress image generated from the contrast agent injection information. The information acquisition unit 70 supplies the acquired information and image to the output unit 80.

(Output Unit 80)

The output unit 80 outputs the slice image acquired by the information acquisition unit 70, and outputs the contrast agent injection information associated with the slice image, the injection progress image generated based on the contrast agent injection information, and the image management information corresponding to the slice image, so as to be superimposed on each other. For example, when the input unit 60 receives a designation of a predetermined time, the output unit 80 outputs a slice image captured at the time that the information acquisition unit 70 has acquired from the storage unit 40 in response to the instruction. In addition, when the input unit 60 receives a designation of a predetermined slice image, the output unit 80 outputs an imaging time, at which the slice image acquired by the information acquisition unit 70 has been acquired, in response to the instruction. The output unit 80 can also output a three-dimensional image together with or separately from the slice image.

In addition, the output unit 80 can also output time series information relevant to the capturing of the slice image as well as the imaging time of the slice image. Specifically, the output unit 80 can output information, which is calculated from the imaging time of each of a plurality of slice images and the slice images, in time series. For example, the output unit 80 can output the time density curve of 2D perfusion (perfusion image) or the time density curve of 3D perfusion as time-series information.

In addition, the medical imaging system 1 can also estimate the position of the blood vessel of the subject at which the contrast agent has arrived, based on the position of the subject at which the contrast agent has been injected and the elapsed time from the injection time of the contrast agent, by segmenting the blood vessels of the subject.

For example, the information acquisition unit 70 segments the blood vessels included in the slice image, and acquires information indicating the position of the subject, at which the contrast agent has been injected, based on the image management information and the contrast agent injection information. Then, the information acquisition unit 70 estimates the arrival position of the contrast agent based on the position, the injection time included in the contrast agent injection information, and the imaging time of the slice image included in the image management information corresponding to the slice image. Then, the output unit 80 can display the injection position of the contrast agent and the arrival position of the contrast agent, which is estimated by the information acquisition unit 70, so as to be superimposed on the slice image. Similarly, the information acquisition unit 70 can calculate the estimated outflow time of the contrast agent for each position of the subject. Then, the output unit 80 can also output the position calculated by the information acquisition unit 70 so as to be superimposed on the slice image.

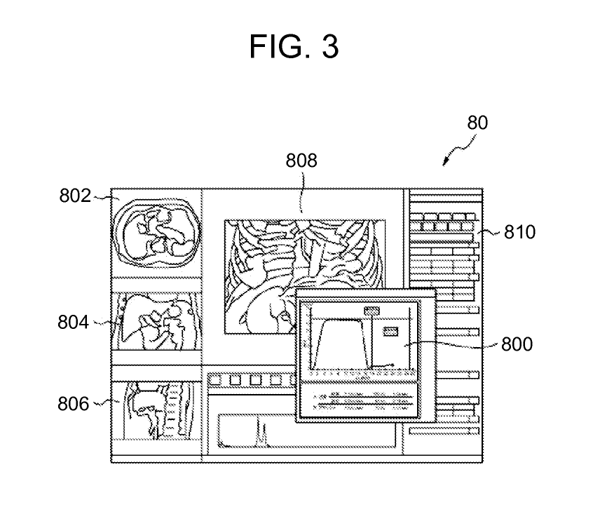

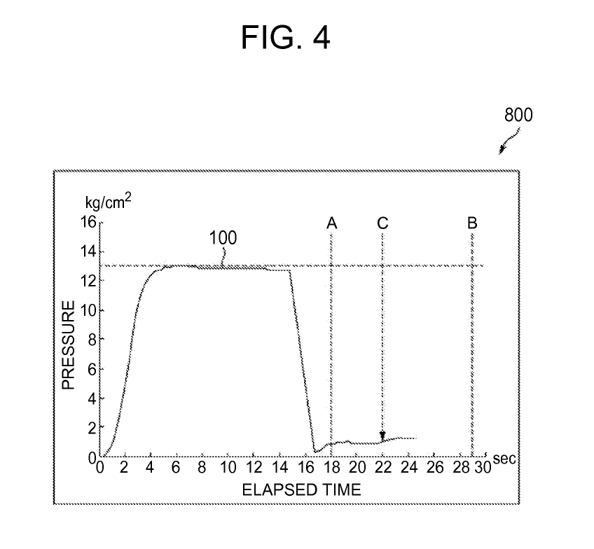

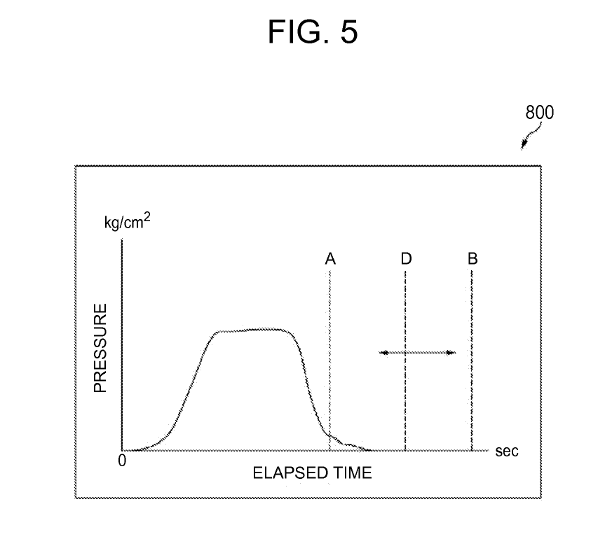

FIG. 3 illustrates an example of an output result in the output unit of the medical imaging system according to the present embodiment, and FIGS. 4 and 5 illustrate an example of an enlarged view of the injection progress image illustrated in FIG. 3.

As illustrated in FIG. 3, the output unit 80 includes a two-dimensional image display region 802 where a two-dimensional slice image of a subject is displayed, a two-dimensional image display region 804 that shows two-dimensional sectional image from reconstructed volume data created from the set of slice images of the subject, another two-dimensional image display region 806 that is reconstructed from a different angle, a three-dimensional image display region 808 where a three-dimensional image created from the volume data of the subject is displayed, and an operation unit display region 810 where an operation unit for designating the execution of predetermined processing for the output two-dimensional sectional image or the output three-dimensional image is displayed.

When a user designates a part of a predetermined image through the input unit 60 for the two-dimensional image or the three-dimensional image that is output to the output unit 80, the information acquisition unit 70 acquires contrast agent injection information associated with the slice image of the designated part and an injection progress image generated based on the contrast agent injection information. Then, the information acquisition unit 70 outputs the contrast agent injection information and the injection progress image to the output unit 80 together with the two-dimensional image display regions 802 to 806, the three-dimensional image display region 808, or the operation unit display region 810.

For example, as illustrated in FIG. 4, an injection progress image 800 has a horizontal axis, which is an elapsed time axis with the injection start time of the contrast agent as the origin, and a vertical axis, which is a pressure axis of the injection pressure of the contrast agent. A graph 100 shows a temporal change in the injection pressure of the contrast agent. In the present embodiment, the output unit 80 outputs the imaging start time “A” and the imaging end time “B” of a plurality of slice images (that is, volume data) in a state of being superimposed on the graph 100 so as to be visible to the user. For example, the output unit 80 outputs such time as a text and/or a figure. In addition, the output unit 80 can also output the injection start time, at which the injection of the contrast agent has started from the contrast agent injection unit 20, and the injection end time, at which the injection of the contrast agent has ended, based on the contrast agent injection information acquired by the information acquisition unit 70.

Between the imaging start time “A” and the imaging end time “B”, the slice image capturing unit 10 has captured a plurality of slice images of the subject. Accordingly, when the user designates a predetermined position (for example, a position “C” in FIG. 4) between the imaging start time “A” and the imaging end time “B”, which are displayed so as to be superimposed on the injection progress image 800, through the input unit 60, the information acquisition unit 70 reads an imaging time at the position from the image management information and outputs the imaging time to the output unit 80. In this case, the information acquisition unit 70 can also acquire a slice image corresponding to the imaging time from the storage unit 40 and output the slice image to the output unit 80. This is because the imaging time changes depending on the part even in a piece of volume data. For example, since a plurality of slice images is captured simultaneously in a multi-slice CT, a plurality of slice images corresponding to the imaging time may be obtained. In this case, a representative image of the plurality of slice images may be output, or all of the plurality of slice images may be output, or an image obtained by combining the plurality of slice images may be output. In addition, a reconstructed image or a three-dimensional image in which a part based on a slice image corresponding to the imaging time is emphasized, of the volume data, may be output. In particular, since a slice image is intermittently captured in the case of performing ECG synchronization or when acquiring a perfusion image, it is meaningful to output the slice image corresponding to the imaging time. When imaging the process of the beating of the heart as a plurality of pieces of volume data after performing ECG synchronization, slice images are alternately captured over the plurality of pieces of volume data. Therefore, it is meaningful to output the slice image corresponding to the imaging time.

In the case of acquiring a plurality of pieces of volume data on the time series, a plurality of imaging start times “A” and a plurality of imaging end times “B” may be present. In this case, when the user designates a predetermined position (for example, a position “C” in FIG. 4) between a certain imaging start time “A” and a certain imaging end time “B” through the input unit 60, the information acquisition unit 70 reads an imaging time at the position from the image management information and outputs the imaging time to the output unit 80. In this case, the information acquisition unit 70 can also acquire volume data corresponding to the imaging time from the storage unit 40 and output the volume data to the output unit 80. More specifically, it is also possible to acquire a slice image corresponding to the imaging time, which is included in volume data corresponding to the imaging time, from the storage unit 40 and output the slice image to the output unit 80. For example, a case can be considered in which a plurality of pieces of volume data is acquired as an arterial phase, a portal phase, and a venous phase in the liver.

That is, as illustrated in FIG. 5, it is possible to designate the imaging time by page-moving a designated position “D” to an arbitrary position between the imaging start time “A” and the imaging end time “B” through the input unit 60. Then, the information acquisition unit 70 can acquire a slice image at the designated imaging time (for example, an imaging time corresponding to the designated position “D”), among the plurality of slice images output to the output unit 80, from the storage unit 40. In the case of volume data, it is possible to acquire a part, which is based on a slice image corresponding to the imaging time, of the volume data.

In this case, the information acquisition unit 70 acquires a plurality of slice images corresponding to the imaging time of a two-dimensional image or a three-dimensional image, which has been designated through the input unit 60, from the storage unit 40, and outputs a slice image satisfying the predetermined criteria to the output unit 80. In addition, the information acquisition unit 70 can acquire contrast agent injection information associated with the slice image from the storage unit 40 and output the contrast agent injection information to the output unit 80 together with the slice image. Therefore, the medical imaging system 1 can provide the user with information that is helpful in understanding the status of the contrast that changes with a specific position in the volume data including a plurality of slice images.

When an instruction to designate a predetermined slice image is input through the input unit 60, the information acquisition unit 70 acquires the imaging time of the slice image from the image management information corresponding to the designated slice image. As illustrated in FIG. 5, the output unit 80 is made to output a sectional imaging time “D” at a position indicating the imaging time in the injection progress image 800.

In this case, in addition to designating a predetermined slice image, it is also possible to designate a part of the three-dimensional image or the reconstructed image. In this case, by acquiring a slice image on which the part is based in the volume data, the imaging time of the slice image on which the part is based is specified. It is also possible to make the output unit 80 output the imaging time as the sectional imaging time “D”.

In addition, while the output unit 80 is outputting a predetermined two-dimensional image or three-dimensional image, the input unit 60 can receive from the user an instruction to display the amount of contrast agent at the imaging time of the image. In response to the instruction received through the input unit 60, the information acquisition unit 70 refers to the image management information corresponding to the slice image that the output unit 80 outputs, and refers to the contrast agent injection information associated with the slice image. Then, the information acquisition unit 70 outputs the amount of contrast agent at the imaging time of the image to the output unit 80 or the injection progress image 800, which is output to the output unit 80, so as to be visible to the user.

(Outline of a Medical Imaging Method)

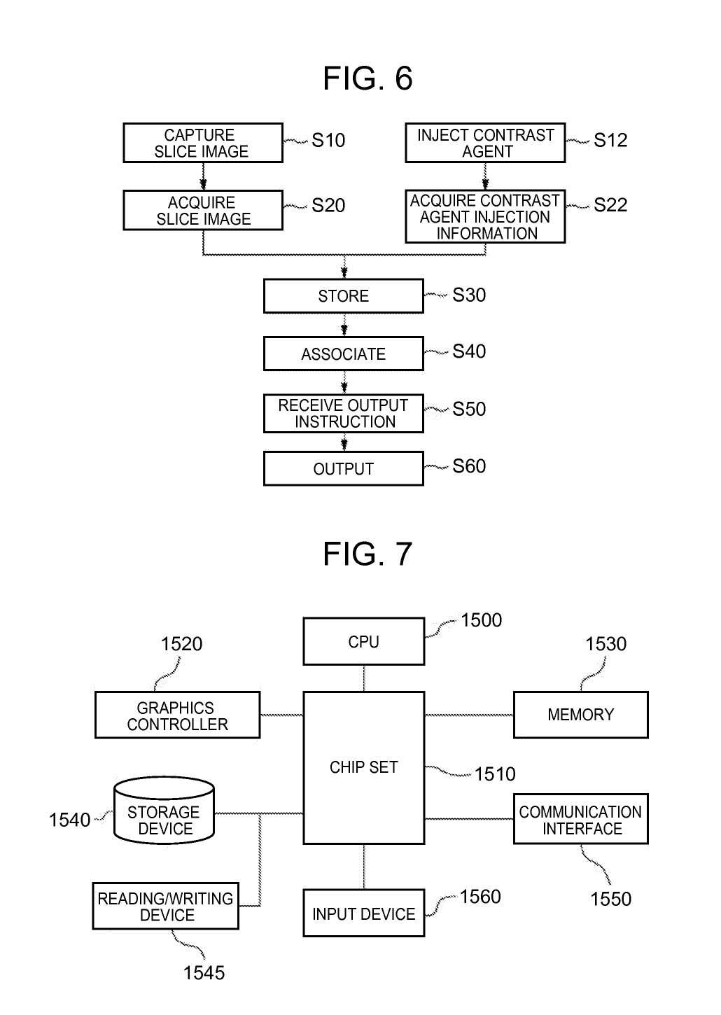

FIG. 6 illustrates an example of the flow of the process of the medical imaging system according to the embodiment of the present invention.

First, the slice image capturing unit 10 captures a set of slice images of the subject (step 10; hereinafter, step will be expressed as “S”). The slice image capturing unit 10 supplies the image data of the captured slice images to the slice image acquisition unit 12. The slice image acquisition unit 12 acquires the image data of the slice images from the slice image capturing unit 10 (S20). The slice image acquisition unit 12 supplies the acquired image data of the slice images to the three-dimensional imaging unit 14, the association constructing unit 30, and the storage unit 40. The three-dimensional imaging unit 14 generates a three-dimensional image using the slice images acquired by the slice image acquisition unit 12. Then, the three-dimensional imaging unit 14 supplies the generated three-dimensional image to the storage unit 40.

On the other hand, the contrast agent injection unit 20 injects a contrast agent into the subject (S12). Then, the injection information acquisition unit 22 acquires contrast agent injection information from the contrast agent injection unit 20 (S22). The injection information acquisition unit 22 supplies the acquired contrast agent injection information to the injection progress imaging unit 24, the association constructing unit 30, and/or the storage unit 40. The injection progress imaging unit 24 generates an injection image using the contrast agent injection information. Then, the injection progress imaging unit 24 supplies the generated injection progress image to the association constructing unit 30 and/or the storage unit 40.

The storage unit 40 stores the image data and the information that have been received from the slice image acquisition unit 12, the three-dimensional imaging unit 14, the injection progress imaging unit 24, and/or the injection information acquisition unit 22 (S30). On the other hand, the association constructing unit 30 compares the image management information (for example, an imaging time) with the contrast agent injection information (for example, a contrast agent injection time), and associates the contrast agent injection information with the slice image (S40). The association constructing unit 30 stores information indicating the association result in the storage unit 40.

Then, when the input unit 60 receives an instruction to output a predetermined image and the contrast agent injection status at a predetermined time (S50), the information acquisition unit 70 acquires the image and the information, which correspond to the instruction received through the input unit 60, from the storage unit 40. Then, the information acquisition unit 70 outputs the acquired image and information to the output unit 80 (S60).

FIG. 7 illustrates an example of the hardware configuration of the medical imaging system according to the embodiment of the present invention.

The medical imaging system 1 according to the present embodiment includes a CPU 1500, a graphics controller 1520, a memory 1530 such as a random access memory (RAM), a read-only memory (ROM), and/or a flash ROM, a storage device 1540 for storing data, a reading/writing device 1545 for reading data from a recording medium and/or for writing data into the recording medium, an input device 1560 for inputting data, a communication interface 1550 for transmitting and receiving data to and from an external communication device, and a chipset 1510 that communicably connects the CPU 1500, the graphics controller 1520, the memory 1530, the storage device 1540, the reading/writing device 1545, the input device 1560, and the communication interface 1550 to each other.

The chipset 1510 executes data transmission between the components by connecting the memory 1530, the CPU 1500 that accesses the memory 1530 to execute predetermined processing, and the graphics controller 1520 that controls the display of an external display device. The CPU 1500 operates based on a program stored in the memory 1530, thereby controlling each component. The graphics controller 1520 displays an image on a predetermined display device based on the image data that is temporarily stored on a buffer provided in the memory 1530.

In addition, the chipset 1510 connects the storage device 1540, the reading/writing device 1545, and the communication interface 1550 to each other. The storage device 1540 stores programs and data that are used in the CPU 1500 of the medical imaging system 1. The storage device 1540 is a flash memory, for example. The reading/writing device 1545 reads a program and/or data from a storage medium in which programs and/or data are stored, and stores the read program and/or data in the storage device 1540. For example, the reading/writing device 1545 acquires a predetermined program from a server on the Internet through the communication interface 1550, and stores the acquired program in the storage device 1540.

The communication interface 1550 executes the transmission and reception of data to and from an external device through a communication network. When a communication network is not available, the communication interface 1550 can also execute the transmission and reception of data to and from an external device without using the communication network. In addition, the input device 1560, such as a keyboard, a tablet, and a mouse, is connected to the chipset 1510 through a predetermined interface.

A program for the medical imaging system 1 stored in the storage device 1540 is provided to the storage device 1540 through a communication network, such as the Internet, or a recording medium, such as a magnetic recording medium or an optical recording medium. The program for the medical imaging system 1 stored in the storage device 1540 is executed by the CPU 1500.

A medical imaging program executed by the medical imaging system 1 according to the present embodiment works on the CPU 1500 to cause the medical imaging system 1 to function as the slice image capturing unit 10, the slice image acquisition unit 12, the three-dimensional imaging unit 14, the contrast agent injection unit 20, the injection information acquisition unit 22, the injection progress imaging unit 24, the association constructing unit 30, the storage unit 40, the information addition unit 50, the input unit 60, the information acquisition unit 70, and the output unit 80, which have been described in FIGS. 1 to 6.

### Effects of the Embodiment

The medical imaging system 1 according to the present embodiment can associate the image data of a slice image with the contrast agent injection information. Therefore, just by designating a slice image that is output to the output unit 80 or a part of the slice image through the input unit 60, information regarding the time, such as the imaging start time of the slice image, the imaging end time of the slice image, and the imaging duration of the slice image, can be output so as to be superimposed on an image showing the injection start time of the contrast agent, the injection duration of the contrast agent, the injection end time of the contrast agent, and a temporal change in the injection pressure of the contrast agent. Therefore, since the medical imaging system 1 can contribute to the estimation of the accuracy of the contrast of the subject and the like, it is possible to contribute to the improvement in the diagnostic accuracy of the subject for the user, such as a doctor.

In addition, the medical imaging system 1 can acquire the image data of a set of slice images as volume data in a single examination, and can store the image management information of each slice image in the storage unit 40 and associate the contrast agent injection information with each slice image. Therefore, since the medical imaging system 1 can separately check the set of slice images captured at different times, it is possible to easily check the injection status of the contrast agent in slice images captured at different imaging times in the same volume data.

In addition, the medical imaging system 1 stores the slice image corresponding to the image management information, the contrast agent injection information, and the injection progress image generated based on the contrast agent injection information, in the storage unit 40. Therefore, any different terminal 6 can refer to the slice image of the subject, the image management information, and the contrast agent injection information just by accessing the storage unit 40, as long as the terminal 6 is communicably connected to the storage unit 40 by cable communication or radio communication. According to the medical imaging system 1, it is possible to generate a graph, which shows a temporal change in the amount of injection of the contrast agent into the patient, from the contrast agent injection information and to centrally manage graph data indicating the graph in the storage unit 40.

In addition, since the medical imaging system 1 can associate the image data of the slice image with the contrast agent injection information and store the result in the storage unit 40, it is not necessary to add a radiology information system (RIS) and other devices. Therefore, as the medical imaging system 1, a system that allows the reference of the slice image and allows the contrast agent injection status at the imaging time of the slice image to be easily checked at a glance can be realized at a low cost.

While the embodiment of the present invention has been described above, the embodiment described above does not limit the invention defined in the appended claims. In addition, it should be noted that all the combinations of the features described in the embodiment are not essential for means for solving the problems of the invention.

### DESCRIPTION OF REFERENCE NUMERALS

1: medical imaging system
2: imaging apparatus
3: injector
4: server
5: injector server
6, 6a, 6n: terminal
10: slice image capturing unit
12: slice image acquisition unit
14: three-dimensional imaging unit
20: contrast agent injection unit
22: injection information acquisition unit
24: injection progress imaging unit
30: association constructing unit
40: storage unit
50: information addition unit
60: input unit
70: information acquisition unit
80: output unit
100: graph
800: injection progress image
802, 804, 806: two-dimensional image display region
808: three-dimensional image display region
810: operation unit display region
1500: CPU
1510: chipset
1520: graphics controller
1530: memory
1540: storage device
1545: reading/writing device
1550: communication interface
1560: input device

1-10. (canceled)

11. A medical imaging system, comprising:
a slice image acquisition unit that acquires a set of slice images of a subject with an image management information for managing the slice images;
an injection information acquisition unit that acquires contrast agent injection information that injected into the subject;
an injection progress image generating unit that generates an injection progress image showing an injection progress of the contrast agent based on the contrast agent injection information;
an association constructing unit that associates the contrast agent injection information with the slice image based on the contrast agent injection information and the image management information; and
an output unit that outputs an slice image from the set of slice images and that outputs the injection progress image generated based on the contrast agent injection information with the image management information corresponding to the slice image.

12. The medical imaging system according to claim 11,
wherein, when a slice image is selected, the output unit outputs an image acquisition time of the slice image.

13. The medical imaging system according to claim 11, further comprising:
an information addition unit that adds the image management information, which corresponds to the slice image associated with the contrast agent injection information, to the contrast agent injection information.

14. The medical imaging system according to any one of claim 11,
wherein the output unit outputs time series information relevant to capturing of the slice image based on the contrast agent injection information, the slice image associated with the contrast agent injection information, and the image management information corresponding to the slice image.

15. The medical imaging system according to any one of claim 11, further comprising:
a storage unit that stores the slice image corresponding to the image management information, the contrast agent injection information, and the injection progress image generated based on the contrast agent injection information.

16. The medical imaging system according to any one of claim 11, further comprising:
a three-dimensional image generating unit that generates a three-dimensional image from the slice image,
wherein the output unit outputs the three-dimensional image.

17. The medical imaging system according to claim 16,
wherein, when a designation of a predetermined time is received from outside, the output unit emphasizes and outputs a portion, which is based on the slice image captured at the time, in the three-dimensional image.

18. The medical imaging system according to claim 16,
wherein, when a designation of a portion of the predetermined three-dimensional image is received from outside, the output unit outputs an imaging time of the slice image on which the portion is based.

19. A medical imaging method in an image processing apparatus, the method comprising:
a slice image acquisition step of acquiring a set of slice images of a subject with an image management information for managing the slice images;
an injection information acquisition step of acquiring contrast agent injection information that injected into the subject;
an injection progress image generating step of generating an injection progress image showing an injection progress of the contrast agent based on the contrast agent injection information;
an association constructing step of associating the contrast agent injection information with the slice image based on the contrast agent injection information and the image management information; and
an output step of outputting an slice image from the set of slice images and outputting the injection progress image generated based on the contrast agent injection information with the image management information corresponding to the slice image.

20. A non-transitory computer-readable storage medium which stores a medical imaging program for causing at least one processor to execute operations including:
a slice image acquisition function of acquiring a set of slice images of a subject with an image management information for managing the slice images;
an injection information acquisition function of acquiring contrast agent injection information that injected into the subject;
an injection progress image generating function of generating an injection progress image showing an injection progress of the contrast agent based on the contrast agent injection information;
an association constructing function of associating the contrast agent injection information with the slice image based on the contrast agent injection information and the image management information; and
an output function of outputting an slice image from the set of slice images and outputting the injection progress image generated based on the contrast agent injection information with the image management information corresponding to the slice image.
